(19) United States
(12) Patent Application Publication
(10) Pub. No.: US 2023/0309845 A1
(43) Pub. Date: Oct. 5, 2023

(54) TOILET SYSTEM

(71) Applicant: TOTO LTD., Kitakyushu-shi (JP)

(72) Inventors: Kenta SUZUKI, Kitakyushu-shi (JP); Tetsuya UCHIDA, Kitakyushu-shi (JP); Teruyuki KAMORI, Kitakyushu-shi (JP); Ryota TAKAHASHI, Kitakyushu-shi (JP); Kaori NISHIJIMA, Kitakyushu-shi (JP); Minoru SATO, Kitakyushu-shi (JP); Yo MOROTOMI, Kitakyushu-shi (JP); Kota YAMASAKI, Kitakyushu-shi (JP); Takashi MATSUURA, Kitakyushu-shi (JP)

(21) Appl. No.: 18/128,606

(22) Filed: Mar. 30, 2023

(30) Foreign Application Priority Data
Mar. 31, 2022 (JP) 2022-059283
Feb. 24, 2023 (JP) 2023-026896

(51) Int. Cl. A61B 5/026; E03D 9/00; A47K 13/30; A61B 5/00

(52) U.S. Cl. CPC A61B 5/026; E03D 9/00; A47K 13/305; A61B 5/6887; A61B 2560/0252; A61B 2560/0276

(57) ABSTRACT

A toilet system includes: a toilet seat having a seat surface on which a user is to sit; a sensor provided in or on the toilet seat, configured to measure a physical quantity which reflects blood flow conditions of the user; a health index calculator configured to calculate a health index of the user based on measurement results of the sensor; a health index output part configured to output the health index of the user which has been calculated by the health index calculator; a temperature determining part configured to determine whether a temperature state of the seat surface satisfies a predetermined condition or not, and an operation change instruction part configured to change an operation of the health index calculator and/or an operation of the health index output part when the temperature determining part determines that the temperature state of the seat surface satisfies the predetermined condition.

### BACKGROUND OF THE INVENTION

### Technical Field

The present invention pertains to a toilet system that can provide a health index of a user.

### Background Art

JP-A-2021-68396 has disclosed a system that measures blood flow conditions of a user who sits on a toilet seat, calculates a health index based on measurement results thereof, and outputs the calculated health index (for example, causes a display terminal to display the calculated health index).

### Patent Document List

JP-A-2021-68396

### SUMMARY OF THE INVENTION

### Technical Problem

The present inventors have found that blood flow conditions of a user who sits on a toilet seat may be greatly affected by a temperature state of the toilet seat. For example, based on a Dynamic Light Scattering method, a laser sensor is attached in or on a toilet seat, which can measure blood flow conditions within a skin on a back side of a user's thigh. Fourier transform or the like is applied to an output signal from the laser sensor so that a health index (including a pulse wave, a blood flow amount, a heart rate, or the like) can be calculated (derived).

Herein, the present inventors have found that, for example on a cold winter day, when a user sits on a cold toilet seat, blood flow conditions of the user who sits on the cold toilet seat may be worsened due to the coldness. A health index calculated based on such worsened blood flow conditions may not correctly reflect the user's actual health status. That is to say, when a temperature of the toilet seat is so low, a health index calculated based on the blood flow conditions of the user who sits on the cold toilet seat may be likely to incorrectly reflect the user's actual health status. For example, an output result of the health index may greatly fluctuate even if the user's actual health status is stable. This may give unnecessary confusion and/or upset to the user who daily takes care of his or her health with reference to the health index obtained via the toilet system. This may undermine credibility as a system for providing a health index. Furthermore, when the temperature of the toilet seat is raised (heated) rapidly within a short period of time, the blood flow conditions of the user who sits on the toilet seat may be greatly affected by the temperature's change. For example, a large change (gradient) may appear in the blood flow conditions of the user. The blood flow conditions measured during such a transition may not correctly reflect the user's actual health status. That is to say, a health index calculated based on such blood flow conditions may not correctly reflect the user's actual health status.

The present invention has been made based on the above findings by the inventors. The object of the present invention is to provide a toilet system which can provide a health index of a user, and which can inhibit that any health index that is likely to incorrectly reflect the user's actual health status is outputted as correctly reflecting the user's actual health status.

### Solution to the Problem

One aspect of the present invention is a toilet system including: a toilet seat having a seat surface on which a user is to sit; a sensor provided in or on the toilet seat, configured to measure a physical quantity which reflects blood flow conditions of the user; a health index calculator configured to calculate a health index of the user based on measurement results of the sensor; a health index output part configured to output the health index of the user which has been calculated by the health index calculator; a temperature determining part configured to determine whether a temperature state of the seat surface satisfies a predetermined condition or not, and an operation change instruction part configured to change an operation of the health index calculator and/or an operation of the health index output part when the temperature determining part determines that the temperature state of the seat surface satisfies the predetermined condition.

According to the above aspect, when the temperature determining part determines that the temperature state of the seat surface satisfies the predetermined condition, an operation of the health index calculator and/or an operation of the health index output part is changed. Specifically, for example on a cold winter day, when it is determined that the temperature of the seat surface is equal to or lower than 20° C., an output of any health index that is likely to incorrectly reflect the user's actual health status is stopped, or such an error message or error information is outputted together with (or instead of) an output of any health index that is likely to incorrectly reflect the user's actual health status, or a calculation operation of the health index calculator is modified depending on the temperature state of the seat surface. Thereby, for example on a cold winter day, when a user sits on a cold toilet seat, it is effectively inhibited that any health index that is likely to incorrectly reflect the user's actual health status is outputted as correctly reflecting the user's actual health status.

Specifically, for example, it is preferable that the temperature determining part may is configured to determine whether the temperature state of the seat surface is equal to or lower than a predetermined first threshold value (for example, 10° C.) or not, and that the operation change instruction part is configured to stop an output operation of the health index output part when the temperature determining part determines that the temperature state of the seat surface is equal to or lower than the first threshold value. According to this manner, any health index that is likely to incorrectly reflect the user's actual health status is stopped to be outputted. Therefore, it is effectively inhibited that unnecessary confusion and/or upset is given to the user who daily takes care of his or her health with reference to the health index obtained via the toilet system.

Alternatively, for example, it is preferable that the temperature determining part is configured to determine whether the temperature state of the seat surface is equal to or lower than a predetermined second threshold value (for example, 10° C.) or not, and that the operation change instruction part is configured to cause the health index output part to output error information when the temperature determining part determines that the temperature state of the seat surface is equal to or lower than the second threshold value. According to this manner, error information (which may be or include an error message) is outputted together with (or instead of) an output of any health index that is likely to incorrectly reflect the user's actual health status. Therefore, it is effectively inhibited that unnecessary confusion and/or upset is given to the user who daily takes care of his or her health with reference to the health indices obtained via the toilet system.

Alternatively, for example, it is preferable that the temperature determining part is configured to determine whether the temperature state of the seat surface is within a predetermined third range (for example, 0° C. to 20° C.) or not, and that the operation change instruction part is configured to modify a calculation operation of the health index calculator (for example, switch over a health index calculation program) when the temperature determining part determines that the temperature state of the seat surface is within the predetermined third range. According to this manner, it is inhibited to calculate any health index that is likely to incorrectly reflect the user's actual health status. Therefore, it is effectively inhibited that unnecessary confusion and/or upset is given to the user who daily takes care of his or her health with reference to the health index obtained via the toilet system.

Information used by the temperature determining part as the basis of determination may be, for example, measurement results of a temperature sensor which is configured to measure a temperature of the seat surface. That is to say, in a preferable embodiment of the present invention, the toilet system further includes a temperature sensor configured to measure a temperature of the seat surface, and the temperature determining part is configured to determine whether the temperature state of the seat surface satisfies the predetermined condition or not based on measurement results of the temperature sensor. According to this manner, a temperature of the seat surface can be directly measured (obtained), which leads to a higher degree of accuracy in a temperature determination (a temperature determining step) by the temperature determining part.

Alternatively, information used by the temperature determining part as the basis of determination may be measurement results of a room temperature sensor which is configured to measure an environment temperature around the seat surface. That is to say, in a preferable embodiment of the present invention, the toilet system further includes a room temperature sensor configured to measure an environment temperature around the seat surface, and the temperature determining part is configured to determine whether the temperature state of the seat surface satisfies the predetermined condition or not based on measurement results of the room temperature sensor. According to this manner, a temperature measurement for the seat surface can be obtained (estimated) accurately to some extent.

Alternatively, information used by the temperature determining part as the basis of determination may be weather information (in particular, forecasted temperature) at a region including a house or facility in which the toilet system is installed. That is to say, in a preferable embodiment of the present invention, the temperature determining part is configured to determine whether the temperature state of the seat surface satisfies the predetermined condition or not based on such weather information. The weather information may be received by the toilet system via the internet and may be updated at a suitable timing. According to this manner as well, a temperature measurement for the seat surface can be obtained (estimated) accurately to some extent.

Alternatively, information used by the temperature determining part as the basis of determination may be an operational state of a heater which is configured to heat the seat surface. Specifically, when the heater is on, a set nominal temperature of the heater (for example, a selected nominal temperature when it is possible to select a plurality of level settings of temperature) may be used as the temperature state of the seat surface in the present invention. That is to say, in a preferable embodiment of the present invention, the toilet system further includes a heater configured to heat the seat surface, and the temperature determining part is configured to determine whether the temperature state of the seat surface satisfies the predetermined condition or not based on an operational state of the heater.

When the heater is off, for example depending on an elapsed time since the heater was turned off, the temperature state of the seat surface may be estimated based on a temperature transition curve forecasted from the set nominal temperature of the heater. When the elapsed time since the heater was turned off is long enough, the other information as described above (the measurement results of the temperature sensor, the measurement results of the room temperature sensor, and/or the weather information) may be used as the basis of determination.

Alternatively, information used by the temperature determining part as the basis of determination may be measurement results of a timer which is configured to measure a seating time during which the user continues to sit on the seat surface. Specifically, when the seating time exceeds for example 10 seconds, 36° C. (an average normal body temperature) may be used as the temperature state of the seat surface in the present invention (even when it is a cold winter day, the seat surface would be warmed by the user's body temperature). That is to say, in a preferable embodiment of the present invention, the toilet system further includes a timer configured to measure a seating time during which the user continues to sit on the seat surface, and the temperature determining part is configured to determine whether the temperature state of the seat surface satisfies the predetermined condition or not based on measurement results of the timer.

Another aspect of the present invention is a toilet system including: a toilet seat having a seat surface on which a user is to sit; a sensor provided in or on the toilet seat, configured to measure a physical quantity which reflects blood flow conditions of the user; a health index calculator configured to calculate a health index of the user based on measurement results of the sensor; a health index output part configured to output the health index of the user which has been calculated by the health index calculator; a temperature determining part configured to determine whether a temperature state of the seat surface satisfies a predetermined condition or not, a heater configured to heat the seat surface, a storage part configured to store information as to whether the user desires an output of the health index or not, and a heater controller configured to change an operational manner of the heater when the temperature determining part determines that the temperature state of the seat surface satisfies the predetermined condition and the storage part stores information that the user desires the output of the health index.

According to the above aspect, when the temperature determining part determines that the temperature state of the seat surface satisfies the predetermined condition and the storage part stores information that the user desires the output of the health index, an operational manner of the heater is changed. Specifically, for example on a cold winter day, when it is determined that the temperature of the seat surface is equal to or lower than 20° C. and the user desires an output of the health index, if the heater has been off, the heater is turned on, or if the heater has been on but a set temperature (or a set temperature level) of the heater has not been sufficient, the set temperature (or the set temperature level) is raised. Thereby, for example on a cold winter day, when a user sits on a cold toilet seat, it is effectively inhibited that any health index that is likely to incorrectly reflect the user's actual health status is outputted as correctly reflecting the user's actual health status.

In addition, a method according to an aspect of the present invention is a method of providing a health index by using a toilet system including: a toilet seat having a seat surface on which a user is to sit; and a sensor provided in or on the toilet seat, configured to measure a physical quantity which reflects blood flow conditions of the user; the method including: a health index calculating step of calculating a health index of the user based on measurement results of the sensor; a health index outputting step of outputting the health index of the user which has been calculated by the health index calculating step; a temperature determining step of determining whether a temperature state of the seat surface satisfies a predetermined condition or not; and an operation change instructing step of changing an operation during the health index calculating step and/or an operation during the health index outputting step when it is determined that the temperature state of the seat surface satisfies the predetermined condition by the temperature determining step.

In addition, a program according to an aspect of the present invention is a program for providing a health index by using a toilet system including: a toilet seat having a seat surface on which a user is to sit; and a sensor provided in or on the toilet seat, configured to measure a physical quantity which reflects blood flow conditions of the user; the program is capable of perform, when executed by a computer: a health index calculating step of calculating a health index of the user based on measurement results of the sensor; a health index outputting step of outputting the health index of the user which has been calculated by the health index calculating step; a temperature determining step of determining whether a temperature state of the seat surface satisfies a predetermined condition or not; and an operation change instructing step of changing an operation during the health index calculating step and/or an operation during the health index outputting step when it is determined that the temperature state of the seat surface satisfies the predetermined condition by the temperature determining step.

In addition, a method according to another aspect of the present invention is a method of providing a health index by using a toilet system including: a toilet seat having a seat surface on which a user is to sit; a sensor provided in or on the toilet seat, configured to measure a physical quantity which reflects blood flow conditions of the user; a heater configured to heat the seat surface; a storage part configured to store information as to whether the user desires an output of the health index or not; the method including: a health index calculating step of calculating a health index of the user based on measurement results of the sensor; a health index outputting step of outputting the health index of the user which has been calculated by the health index calculating step; a temperature determining step of determining whether a temperature state of the seat surface satisfies a predetermined condition or not; and a heater controlling step of changing an operational manner of the heater when it is determined that the temperature state of the seat surface satisfies the predetermined condition by the temperature determining step and the storage part stores information that the user desires the output of the health index.

In addition, a program according to another aspect of the present invention is a program for providing a health index by using a toilet system including: a toilet seat having a seat surface on which a user is to sit; a sensor provided in or on the toilet seat, configured to measure a physical quantity which reflects blood flow conditions of the user; a heater configured to heat the seat surface; a storage part configured to store information as to whether the user desires an output of the health index or not; the program is capable of perform, when executed by a computer: a health index calculating step of calculating a health index of the user based on measurement results of the sensor; a health index outputting step of outputting the health index of the user which has been calculated by the health index calculating step; a temperature determining step of determining whether a temperature state of the seat surface satisfies a predetermined condition or not; and a heater controlling step of changing an operational manner of the heater when it is determined that the temperature state of the seat surface satisfies the predetermined condition by the temperature determining step and the storage part stores information that the user desires the output of the health index.

A further other aspect of the present invention is a toilet system including: a toilet seat having a seat surface on which a user is to sit; a sensor provided in or on the toilet seat, configured to measure a physical quantity which reflects blood flow conditions of the user; a heater configured to heat the seat surface; and a heater controller configured to control an operational manner of the heater while the sensor measures the physical quantity. According to the above aspect, while the sensor measures the physical quantity, the heater controller controls an operational manner of the heater so that the temperature state of the seat surface on which the user sits can be brought close to or maintained within a desired temperature range, for example with a higher degree of accuracy. Specifically, for example in comparison with a room temperature of a toilet room, when an operational manner of a heater is too weak (which may happen when an energy saving mode is set (used)), blood flow conditions of an user who sits on a toilet seat including (heated by) the heater may be affected (changed) by the seat surface because the seat surface's temperature would be lower than the user's body temperature. On the other hand, when an operational manner of a heater is too strong (which may happen when a rapid heating mode is set (used)), blood flow conditions of a user who sits on a toilet seat including (heated by) the heater may be affected (changed) by the seat surface because the seat surface's temperature would be higher than the user's body temperature. Furthermore, it is predicted that a temperature range which the seat surface should be in (satisfy) for measuring blood flow conditions of a user correctly may be stricter than a temperature range which the seat surface should be in (satisfy) for not making uncomfortable the user who sits on the seat surface. In such a case, it is very effective that the heater controller controls an operational manner of the heater while the sensor measures the physical quantity in such a manner that the seat surface satisfies the latter temperature range (for example, in such a manner that the seat surface's temperature is brought closer to the user's body temperature).

### Advantageous Effects of Invention

According to one aspect of the present invention, when the temperature determining part determines that the temperature state of the seat surface satisfies the predetermined condition, an operation of the health index calculator and/or an operation of the health index output part is changed. Specifically, for example on a cold winter day, when it is determined that the temperature of the seat surface is equal to or lower than 20° C., an output of any health index that is likely to incorrectly reflect the user's actual health status is stopped, or such an error message or error information is outputted together with (or instead of) an output of any health index that is likely to incorrectly reflect the user's actual health status, or a calculation operation of the health index calculator is modified depending on the temperature state of the seat surface. Thereby, for example on a cold winter day, when a user sits on a cold toilet seat, it is effectively inhibited that any health index that is likely to incorrectly reflect the user's actual health status is outputted as correctly reflecting the user's actual health status.

Alternatively, according to another aspect of the present invention, when the temperature determining part determines that the temperature state of the seat surface satisfies the predetermined condition and the storage part stores information that the user desires the output of the health index, an operational manner of the heater is changed. Specifically, for example on a cold winter day, when it is determined that the temperature of the seat surface is equal to or lower than 20° C. and the user desires an output of the health index, if the heater has been off, the heater is turned on, or if the heater has been on but a set temperature (or a set temperature level) of the heater has not been sufficient, the set temperature (or the set temperature level) is raised. Thereby, for example on a cold winter day, when a user sits on a cold toilet seat, it is effectively inhibited that any health index that is likely to incorrectly reflect the user's actual health status is outputted as correctly reflecting the user's actual health status.

Alternatively, according to a further other aspect of the present invention, while the sensor measures the physical quantity, the heater controller controls an operational manner of the heater so that the temperature state of the seat surface on which the user sits can be brought close to or maintained within a desired temperature range, for example with a higher degree of accuracy.

### BRIEF DESCRIPTION OF DRAWINGS

FIG. 1 is a schematic perspective view of a toilet system according to an embodiment of the present invention,

FIG. 2 is an exploded perspective view of a toilet seat of the toilet system shown in FIG. 1,

FIG. 3 is a schematic block view showing main components of the toilet system shown in FIG. 1,

FIG. 4 is a schematic view showing a structure of a laser sensor of the toilet system shown in FIG. 1,

FIG. 5 is a schematic view showing a process for calculating various health indices based on measurement results of the laser sensor shown in FIG. 4,

FIG. 6 is a flowchart showing a first operation example of the toilet system shown in FIG. 1,

FIG. 7 is a flowchart showing a second operation example of the toilet system shown in FIG. 1,

FIG. 8 is a flowchart showing a third operation example of the toilet system shown in FIG. 1, and

FIG. 9 is a flowchart showing a variant of the third operation example of the toilet system shown in FIG. 1.

### DETAILED DESCRIPTION OF THE EMBODIMENTS

### Structure

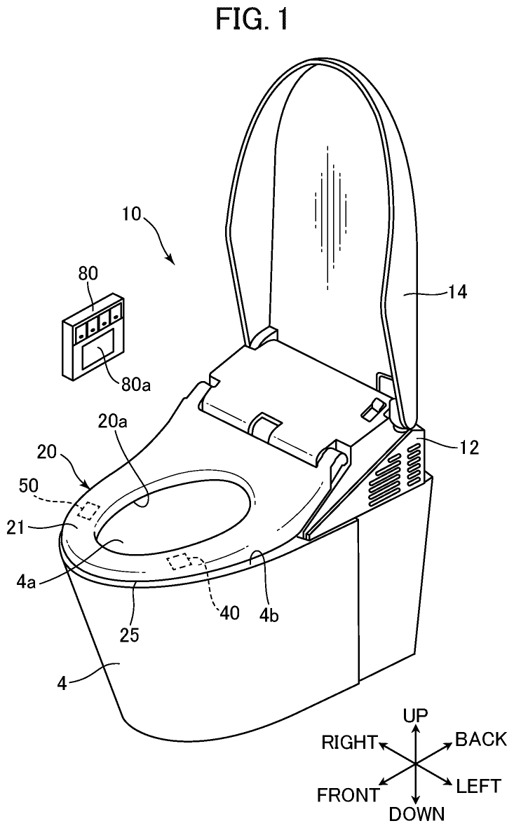

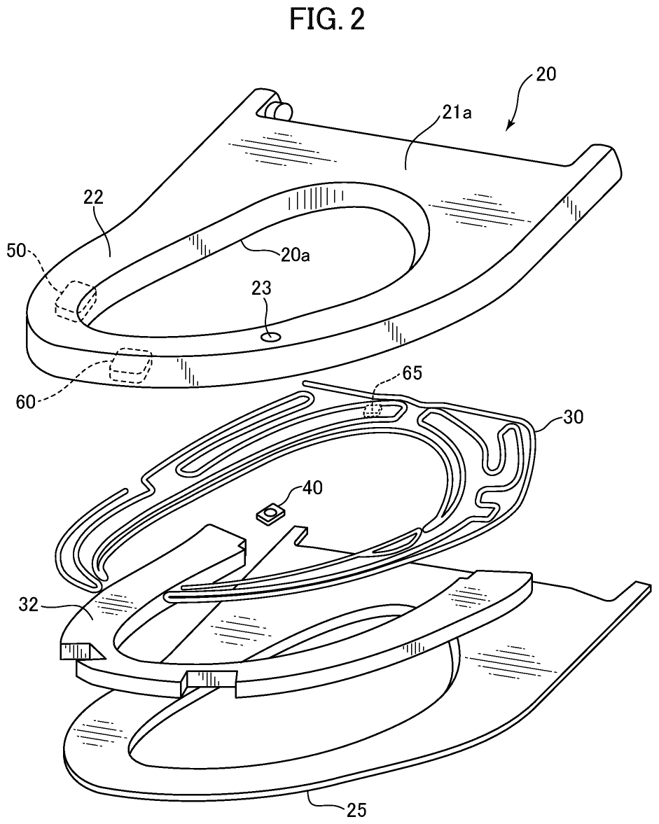

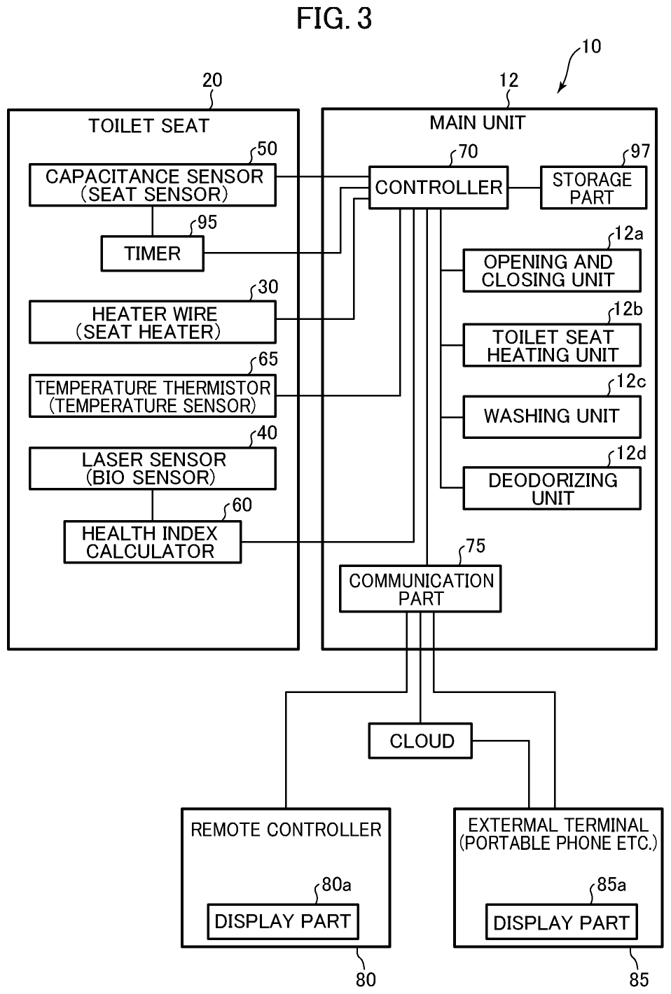

Hereinafter, an embodiment of the present invention is explained with reference to the attached drawings. FIG. 1 is a schematic perspective view of a toilet system 10 according to an embodiment of the present invention, FIG. 2 is an exploded perspective view of a toilet seat 20 of the toilet system 10 shown in FIG. 1, and FIG. 3 is a schematic block view showing main components of the toilet system 10 shown in FIG. 1.

As shown in FIGS. 1 to 3, the toilet system 10 of the present embodiment includes a toilet seat 20, a main unit 12 and a toilet cover 14. The toilet seat 20 and the toilet cover 14 are supported by the main unit 12 in a pivotable manner with respect to the main unit 12.

A laser sensor 40 is provided as a sensor that measures a physical quantity which reflects blood flow conditions of a user. A capacitance sensor 50 is provided as a seat sensor (seat occupancy sensor). A health index calculator 60 (specifically, for example, a microprocessor), which is configured to calculate a plurality of health indices of the user based on measurement results by the laser sensor 40, is provided in the toilet seat 20. Furthermore, a temperature thermistor 65 (an example of a temperature sensor), which is configured to measure a temperature of the toilet seat 20, is provided in the toilet seat 20 of the present embodiment.

The toilet seat 20 has an opening part 20a. In the present embodiment, an O-shaped opening 20a is formed in a central area of the toilet seat 20. The opening of the toilet seat 20 is not limited to such an O-shaped opening, but also may be a U-shaped opening. An outer periphery of the toilet seat 20 is curved along an outside contour of a toilet bowl unit 4. The toilet seat 20 is generally made of opaque resin (for example, polypropylene), and has a seat surface 21 on which a user is to sit and has a bottom surface 25 opposite to the seat surface 21.

The seat surface 21 is a surface exposed upward under a state wherein the toilet seat 20 is placed on an upper surface 4b of the toilet bowl unit 4 (wherein the toilet seat 20 is lowered). The bottom surface 25 is a surface facing to the upper surface 4b of the toilet bowl unit 4 under the same state wherein the toilet seat 20 is placed on the upper surface 4b of the toilet bowl unit 4 (wherein the toilet seat 20 is lowered). The substantially whole of the toilet seat 20 is consists of a thick-walled portion 22, but a thin-walled portion 23, which is thinner than the thick-walled part 22, is formed locally at a position which the laser sensor 40 corresponds to.

As shown in FIG. 2, a heater wire 30 (an example of a heater) and an insulator 32 are provided in the toilet seat 20 to heat or keep warm the seat surface 21. The heater wire 30 is configured to be controlled by a toilet seat heating unit 12b provided in the main part 12, and is stretched around the inside of the toilet seat 20 so as not to interfere with the laser sensor 40, the capacitance sensor 50, the health index calculator 60 and the temperature thermistor 65. The insulator 32 is arranged below the heater wire 30, the laser sensor 40, the capacitance sensor 50, the health index calculator 60 and the temperature thermistor 65.

The thin-walled portion 23 is so thin that an irradiated light from the laser sensor 40 and a reflected light from the user who sits on the seat surface 21 are transmissive therethrough. The thickness of the thin-walled portion 23 is set according to intensity of the irradiated light from the laser sensor 40 and intensity of the reflected light from the user, and according to durability of the toilet seat 20, and the like. For example, the thickness of the thin-walled portion 23 is about 0.5 mm to 1.0 mm.

As used herein, the terms “up”, “down”, “front”, “back”, “left” and “right” refer to directions viewed from a user sitting on the toilet seat 20 with his or her back facing to the opened toilet cover 14, respectively.

As shown in FIG. 2, the thin-walled portion 23 is formed on the left side and on the front side than the center of the length of the opening 20a of the toilet seat 20 in the front-back direction. That is to say, the thin-walled portion 23 is located on the left side forward from the center of gravity of a user who sits on the toilet seat 20. Thereby, the thin-walled portion 23 faces to (abuts to) a skin on a back side of the user's left thigh.

The thin-walled portion 23 is formed as small as possible to the extent that the laser sensor 40 can detect blood flow information of the user who sits on the toilet seat 20. For example, the thin-walled portion 23 has a circular shape with a diameter of 12 mm or less (preferably, 8 mm or less).

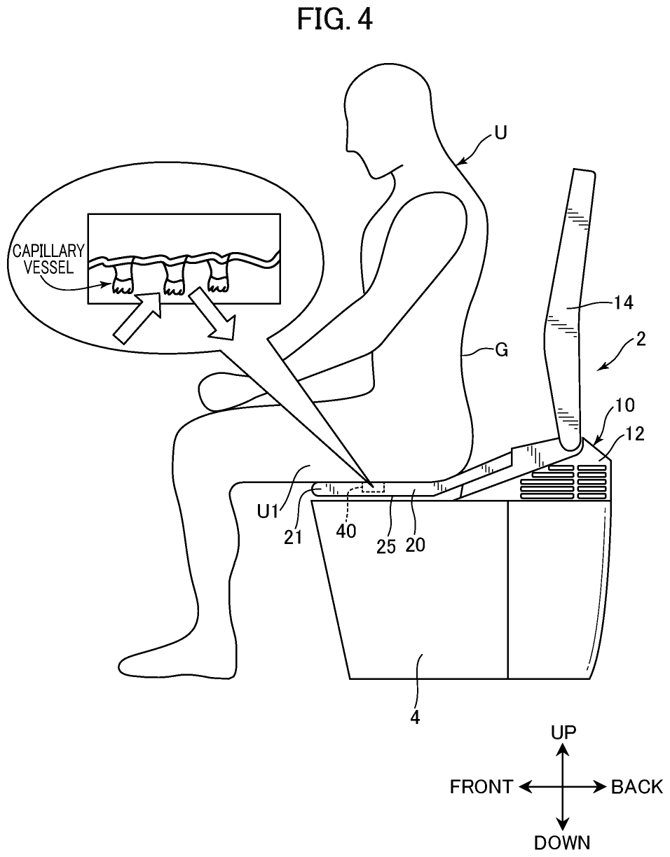

The laser sensor 40 is located on a back side of the thin-walled portion 23 in the toilet seat 20. The laser sensor 40 is a reflective type of sensor that emits an infrared irradiation light toward the back side of the user's left thigh and detects a reflected light (a scattered light resulting from Doppler Shift by red blood cells) that has been reflected according to blood flow conditions in a subcutaneous blood vessel. FIG. 4 is a schematic view showing a structure of the laser sensor 40.

On the other hand, as shown in FIGS. 1 and 2, the capacitance sensor 50 is arranged on the right side and on the front side than the center of the length of the opening 20a of the toilet seat 20 in the front-back direction. That is to say, the capacitance sensor 50 is located on the right side forward from the center of gravity of a user who sits on the toilet seat 20. Thereby, the capacitance sensor 50 faces to (abuts to) a skin on a back side of the user's right thigh, and then the capacitance sensor 50 can detect the user's sitting state (seat occupancy).

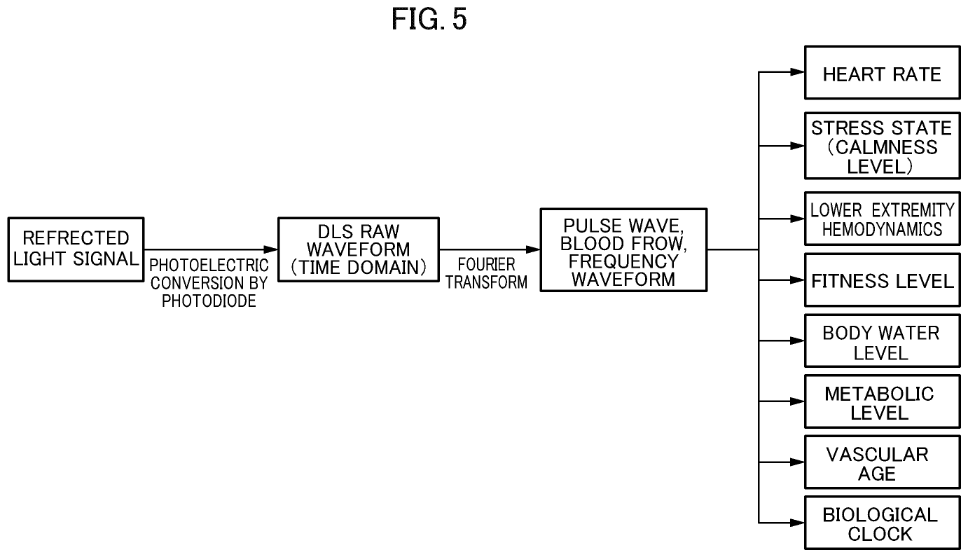

In the present embodiment, the health index calculator 60 is arranged in the vicinity of a front end of the toilet seat 20 (at a position relatively close to the laser sensor 40), and is configured to process an output signal of the laser sensor 40 and transform it to a noise-resistant signal. Specifically, the health index calculator 60 is configured to calculate a plurality of health indices (specifically, for example, a pulse rate, a pulse variability, a blood flow amount) of the user who sits on the toilet seat 20 based on the measurement results of the laser sensor 40, and to transmit a signal corresponding to the calculated results to a communication part 75 via a controller 70. FIG. 5 is a schematic view showing a process for calculating various health indices based on the measurement results of the laser sensor 40.

The controller 70 and the communication part 75 are arranged in the main unit 12. The health index calculator 60 may be arranged in the main unit 12, instead of in the toilet seat 20. When the health index calculator 60 is arranged in the main unit 12, the health index calculator 60 may be provided separately from the controller 70 or integrated with the controller 70 as a part thereof.

In addition, the health index calculator 60 and the controller 70 may be provided (set up) in an external device or an external network (for example, a cloud network) communicable via the communication part 75, instead of in the main unit 12.

With reference to FIG. 1 again, the main unit 12 is attached on the upper surface 4b of the toilet bowl unit 4 on the back side than a bowl portion of the toilet bowl unit 4. An opening and closing unit 12a configured to control an opening and losing operation for each of the toilet seat 20 and cover 14, a toilet seat heating unit 12b configured to control a temperature of the toilet seat 20, a washing unit 12c configured to wash a portion of the user's body, and a deodorizing unit 12d configured to reduce odorous components, are arranged in the main unit 12. The respective units 12a to 12d are controlled by the controller 70 comprehensively. The controller 70 is also connected to each of the capacitance sensor 50 and the temperature thermistor 65.

The controller 70 of the present embodiment is connected to the communication part 75 (an example of a health index output unit) for outputting the plurality of health indices of the user which has been calculated by the health index calculator 60. The communication part 75 is configured to transmit the calculated health indices of the user to, for example, a remote controller 80 in the toilet room or a portable terminal 85 of the user. Thereby, the user who sits on the toilet seat 20 can check various health indices (vital signs such as a pulse rate, etc.) by a display part 80a of the remote controller 80 and/or by a display part 85a of the portable terminal 85.

Herein, as described above, according to the present inventors' findings, for example on a cold winter day, when a user sits on the cold toilet seat 20, blood flow conditions of the user who sits on the cold toilet seat 20 may be worsened due to the coldness, and thus a health index calculated based on such worsened blood flow conditions may not correctly reflect the user's actual health status.

In order to solve this problem, the controller 70 in the toilet system 10 of the present embodiment is configured to determine whether a temperature state of the seat surface 21 satisfies a predetermined condition or not (as a temperature determining part), and to change an operation of the health index calculator 60 and/or an operation of the communication part 75 (health index output part) when it is determined that the temperature state of the seat surface 21 satisfies the predetermined condition (as an operation change instruction part).

Besides, the toilet system 10 of the present embodiment has a timer 95 configured to measure a seating time during which the user continues to sit on the seat surface 21 by cooperating with the capacitance sensor 50. The timer 95 is also connected to the controller 70.

### First Operation Example: Details of Structure

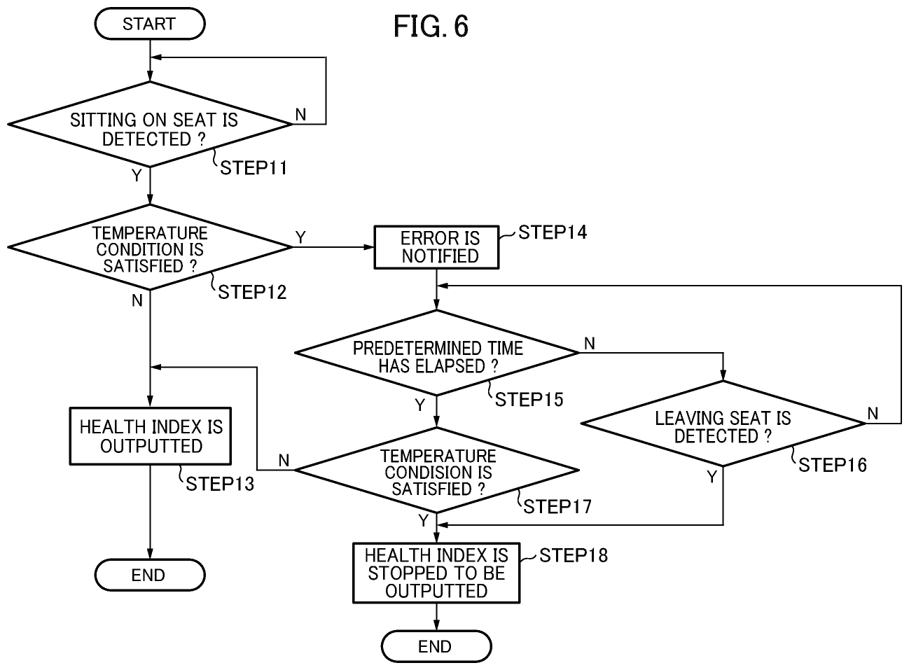

A first operation example of the toilet system 10 as described above is explained with reference to FIG. 6. In the first operation example, the controller 70 is configured to determine whether the user sits (continues to sit) on the seat surface 21, based on the measurement results of the capacitance sensor 50 (an example of a seat sensor), as a seat occupancy determining part. Furthermore, in the first operation example, the controller 70 is configured to determine whether the seating time (elapsed time since the user sat on the seat surface 21) is not shorter than 10 seconds, based on the measurement results of the timer 95, as a seating time determining part.

On the other hand, in the first operation example, the controller 70 is configured to determine whether the temperature state of the seat surface 21 is not higher than 20° C. (an example of a first threshold value and an example of a second threshold value), based on the measurement results of the temperature thermistor 65 (an example of a temperature sensor), as a temperature determining part. Furthermore, in the first operation example, when it is determined that the temperature state of the seat surface 21 is equal to or lower than 20° C. (not higher than 20° C.), the controller 70 is configured to cause the communication part 75 (health index output part) to output error information and/or to stop outputting the health index, as an operation change instruction part.

The reference of the health index being stopped to be outputted means that the health index calculated based on newly obtained blood flow information (measurement results) is stopped to be outputted. For example, the health index previously obtained under a state wherein a user had sat on the seat surface 21 last time may continue to be outputted. That is to say, the reference of the health index being stopped to be outputted may include that the health index which has been previously calculated is not newly updated.

### First Operation Example: Operation

With reference to FIG. 6, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 determines whether the temperature state of the seat surface 21 is not higher than 20° C. (an example of a second threshold value) (STEP 12). In parallel, a calculation process for a plurality of health indices of the user is started by the health index calculator 60 based on measurement results of the laser sensor 40.

When it is determined that the temperature state of the seat surface 21 is higher than 20° C. (NO at STEP 12), a normal operation is carried out. That is to say, the communication part 75 transmits the health indices of the user calculated by the health index calculator 60 to, for example, the remote controller 80 in the toilet room or the portable terminal 85 of the user (STEP 13).

When it is determined that the temperature state of the seat surface 21 is not higher than 20° C. (YES at STEP 12), the controller 70 causes the communication part 75 (health index output part) to output error information (STEP 14). Subsequently, the controller 70 determines whether the seating time (elapsed time since the user sat on the seat surface 21) has reached or exceeded 10 seconds (YES) or has not reached 10 seconds yet (NO) (STEP 15).

Before the seating time reaches 10 seconds (NO at STEP 15), if the capacitance sensor 50 detects the user's leaving the seat surface 21 (YES at STEP 16), the controller 70 causes the communication part 75 (health index output part) to stop outputting the health index (STEP 18).

Before the seating time reaches 10 seconds (NO at STEP 15), while the capacitance sensor 50 does not detect the user's leaving the seat surface 21 (NO at STEP 16), it is waited for the seating time to reach or exceed 10 seconds (NO at STEP 16).

After the seating time has reached or exceeded 10 seconds (YES at STEP 15), the controller 70 determines whether the temperature state of the seat surface 21 is not higher than 20° C. (an example of a first threshold value), again in the present embodiment, based on the measurement results of the temperature thermistor 65 (an example of a temperature sensor) (STEP 17).

When it is determined that the temperature state of the seat surface 21 is higher than 20° C. (NO at STEP 17), a normal operation is carried out. That is to say, the communication part 75 transmits the health indices of the user calculated by the health index calculator 60 to, for example, the remote controller 80 in the toilet room or the portable terminal 85 of the user (STEP 13).

When it is determined that the temperature state of the seat surface 21 is not higher than 20° C. (YES at STEP 17), the controller 70 causes the communication part 75 (health index output part) to stop outputting the health index (STEP 18).

### First Operation Example: Effect

According to the first operation example as described above, for example on a cold winter day, when it is determined that the temperature of the seat surface 21 is equal to or lower than 20° C., error information is outputted from the communication part 75 while the user sits on the seat surface 21, and after the user has left the seat surface 21, the health index is stopped to be outputted from the communication part 75. Thereby, it is effectively inhibited that any health index that is likely to incorrectly reflect the user's actual health status is outputted as correctly reflecting the user's actual health status.

In particular, after the user has left the seat surface 21, since any health index that is likely to incorrectly reflect the user's actual health status is not outputted, it is effectively inhibited that unnecessary confusion and/or upset is given to the user who daily takes care of his or her health with reference to the health index obtained via the toilet system 10.

Furthermore, according to the first operation example, while the user sits on the seat surface 21, error information is outputted together with any health index that is likely to incorrectly reflect the user's actual health status. Thereby, it is effectively inhibited that unnecessary confusion and/or upset is given to the user who daily takes care of his or her health with reference to the health index obtained via the toilet system 10. In addition, according to the first operation example, since a temperature of the seat surface 21 is directly measured (obtained), accuracy in a temperature determination is higher.

### First Operation Example: Variant

Information used as the basis of temperature determination is not limited to the measurement results of the temperature thermistor 65. For example, a room temperature sensor configured to measure a temperature of the toilet room (an environment temperature around the seat surface 21) may be provided, and if the heater configured to heat the seat surface 21 is off, the controller may determine (estimate) whether the temperature state of the seat surface 21 satisfies the predetermined condition or not based on measurement results of the room temperature sensor.

Alternatively, information used as the basis of temperature determination may be a combination of an operational state of a heater (specifically, heater wire 30 in the present embodiment) configured to heat the seat surface 21 and weather information (in particular, forecasted temperature) at a region including a house or facility in which the toilet system 10 is installed. The weather information may be received by the controller 70 of the toilet system 10 via the internet and may be updated at a suitable timing, for example by means of a suitable IOT technology.

Alternatively, information used as the basis of temperature determination may be an operational state of a heater (specifically, heater wire 30 in the present embodiment) configured to heat the seat surface 21. Specifically, when the heater is on, a set nominal temperature of the heater (for example, a selected nominal temperature when it is possible to select a plurality of level settings of temperature) may be used as the temperature state of the seat surface 21 in temperature determination. Furthermore, when the heater is off, for example depending on an elapsed time since the heater was turned off, the temperature state of the seat surface 21 may be estimated based on a temperature transition curve forecasted from the set nominal temperature of the heater. In this case, when the elapsed time since the heater was turned off is long enough, the other information as described above (the measurement results of the temperature sensor, the measurement results of the room temperature sensor, and/or the weather information) may be used as the basis of determination.

Alternatively, information used as the basis of temperature determination may be measurement results of the timer 95 configured to measure the seating time during which the user continues to sit on the seat surface 21. Specifically, when the seating time exceeds for example 10 seconds, 36° C. (an average normal body temperature) may be used as the temperature state of the seat surface 21 in temperature determination (even when it is a cold winter day, the seat surface 21 would be warmed by the user's body temperature). That is to say, in the first operation example shown in FIG. 5, if YES is determined at STEP 15, automatically YES may be determined at STEP 17 as well.

### Second Operation Example: Details of Structure

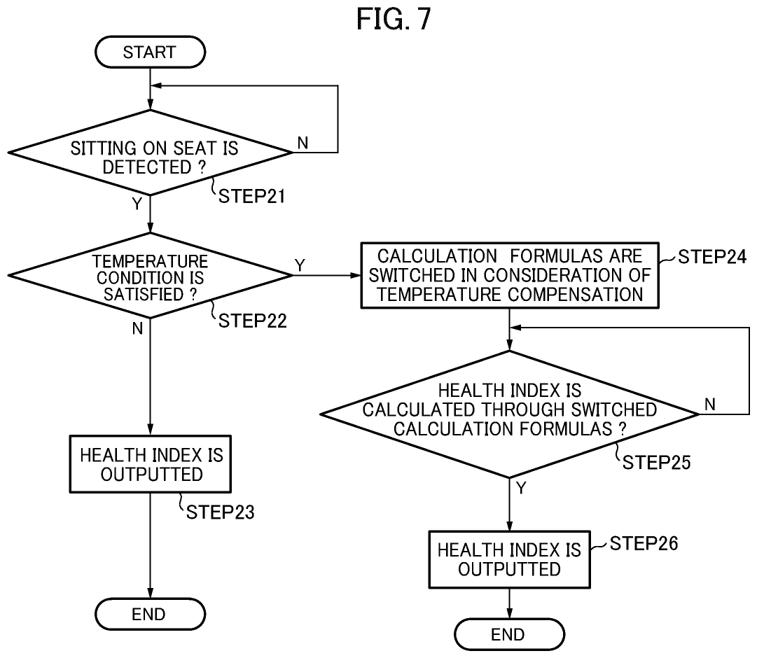

A second operation example of the toilet system 10 as described above is explained with reference to FIG. 7. In the second operation example as well, the controller 70 is configured to determine whether the user sits (continues to sit) on the seat surface 21, based on the measurement results of the capacitance sensor 50 (an example of a seat sensor), as a seat occupancy determining part.

In addition, in the second operation example, the controller 70 is configured to determine whether the temperature state of the seat surface 21 is: within a range of 0° C. to 10° C. (an example of a third range); or within another range of 10° C. to 20° C. (another example of a third range); or outside these ranges (i.e., equal to or higher than 20° C.); based on the measurement results of the temperature thermistor 65 (an example of a temperature sensor), as a temperature determining part.

Then, in the second operation example, when it is determined that the temperature state of the seat surface 21 is within the range of 0° C. to 10° C. or within the range of 10° C. to 20° C., the controller 70 is configured to change an operation of the health index calculator 60, as an operation change instruction part. Specifically, calculation formulas used in the calculation process for calculating a plurality of health indices from the output signal of the laser sensor 40 (see FIG. 5) are switched from those for normal temperature (20° C. or higher) to those for low temperature (10° C. to 20° C.) or to those for very low temperature (0° C. to 10° C.). (The calculation formulas for low temperature (10° C. to 20° C.) and the calculation formulas for very low temperature (0° C. to 10° C.) have been prepared in advance in the health index calculator 60 in a switchable manner.)

### Second Operation Example: Operation

With reference to FIG. 7, the controller 70 determines whether a user sits on the seat surface 21 or not (STEP 21). When it is determined that no user has sat on the seat surface 21 yet (NO at STEP 21), this determining step is repeatedly performed.

When it is determined that a user has sat on the seat surface 21 (YES at STEP 21), the controller 70 determines whether the temperature state of the seat surface 21 is: within the range of 0° C. to 10° C. (an example of a third range); or within the range of 10° C. to 20° C. (another example of a third range); or outside these ranges (i.e., equal to or higher than 20° C.) (STEP 22). In parallel, a calculation process for a plurality of health indices of the user is started by the health index calculator 60 based on measurement results of the laser sensor 40.

When it is determined that the temperature state of the seat surface 21 is outside the set ranges (i.e., is equal to or higher than 20° C.) (NO at STEP 22), a normal operation is carried out. That is to say, the communication part 75 transmits the health indices of the user calculated by the health index calculator 60 to, for example, the remote controller 80 in the toilet room or the portable terminal 85 of the user (STEP 23).

When it is determined that the temperature state of the seat surface 21 is within the range of 0° C. to 10° C. or within the range of 10° C. to 20° C. (YES at STEP 22), the controller 70 controls the health index calculator 60 to change the operation of the health index calculator 60 (STEP 24). Specifically, in the health index calculator 60, calculation formulas used in the calculation process for calculating a plurality of health indices from the output signal of the laser sensor 40 (see FIG. 5) are switched from those for normal temperature (20° C. or higher) to those for low temperature (10° C. to 20° C.) or to those for very low temperature (0° C. to 10° C.).

Subsequently, by the health index calculator 60 in which the calculation formulas have been switched, the calculation process for calculating a plurality of health indices of the user is carried out (executed) once again (a rest may be done before the calculation process if needed). Then, the controller 70 confirms that the calculated health indices have been obtained through the switched calculation formulas (through the changed operation) (STEP 25).

Before the confirmation result is affirmed (NO at STEP 25), it is waited for the confirmation result to be affirmed (NO at STEP 25).

After the confirmation result has been affirmed (YES at STEP 25), the communication part 75 transmits the health indices of the user calculated by the health index calculator 60 using the switched calculation formulas (by the changed operation) to, for example, the remote controller 80 in the toilet room or the portable terminal 85 of the user (STEP 26).

### Second Operation Example: Effect

According to the second operation example as described above, for example on a cold winter day, when the temperature of the seat surface 21 is within the range of 0° C. to 10° C. or within the range of 10° C. to 20° C., the calculation formulas used in the calculation process for calculating a plurality of health indices from the output signal of the laser sensor 40 (see FIG. 5) are switched from those for normal temperature (20° C. or higher) to those for low temperature (10° C. to 20° C.) or to those for very low temperature (0° C. to 10° C.). Thereby, it is effectively inhibited that any health index that is likely to incorrectly reflect the user's actual health status is outputted as correctly reflecting the user's actual health status.

In addition, according to the second operation example as well, since a temperature of the seat surface 21 is directly measured (obtained), accuracy in a temperature determination is higher.

### Second Operation Example: Variant

In the second operation example as well, information used as the basis of temperature determination is not limited to the measurement results of the temperature thermistor 65. For example, a room temperature sensor configured to measure a temperature of the toilet room (an environment temperature around the seat surface 21) may be provided, and if the heater configured to heat the seat surface 21 is off, the controller may determine (estimate) whether the temperature state of the seat surface 21 satisfies the predetermined condition or not based on measurement results of the room temperature sensor.

Alternatively, information used as the basis of temperature determination may be a combination of an operational state of a heater (specifically, heater wire 30 in the present embodiment) configured to heat the seat surface 21 and weather information (in particular, forecasted temperature) at a region including a house or facility in which the toilet system 10 is installed. The weather information may be received by the toilet system 10 via the internet and may be updated at a suitable timing, for example by means of a suitable IOT technology.

Alternatively, information used as the basis of temperature determination may be an operational state of a heater (specifically, the heater wire 30 in the present embodiment) configured to heat the seat surface 21. Specifically, when the heater is on, a set nominal temperature of the heater (for example, a selected nominal temperature when it is possible to select a plurality of level settings of temperature) may be used as the temperature state of the seat surface 21 in temperature determination. Furthermore, when the heater is off, for example depending on an elapsed time since the heater was turned off, the temperature state of the seat surface 21 may be estimated based on a temperature transition curve forecasted from the set nominal temperature of the heater. In this case, when the elapsed time since the heater was turned off is long enough, the other information as described above (the measurement results of the temperature sensor, the measurement results of the room temperature sensor, and/or the weather information) may be used as the basis of determination.

Alternatively, information used as the basis of temperature determination may be measurement results of the timer 95 configured to measure the seating time during which the user continues to sit on the seat surface 21. Specifically, when the seating time exceeds for example 10 seconds, 36° C. (an average normal body temperature) may be used as the temperature state of the seat surface 21 in temperature determination (even when it is a cold winter day, the seat surface 21 would be warmed by the user's body temperature).

### Third Operation Example: Details of Structure

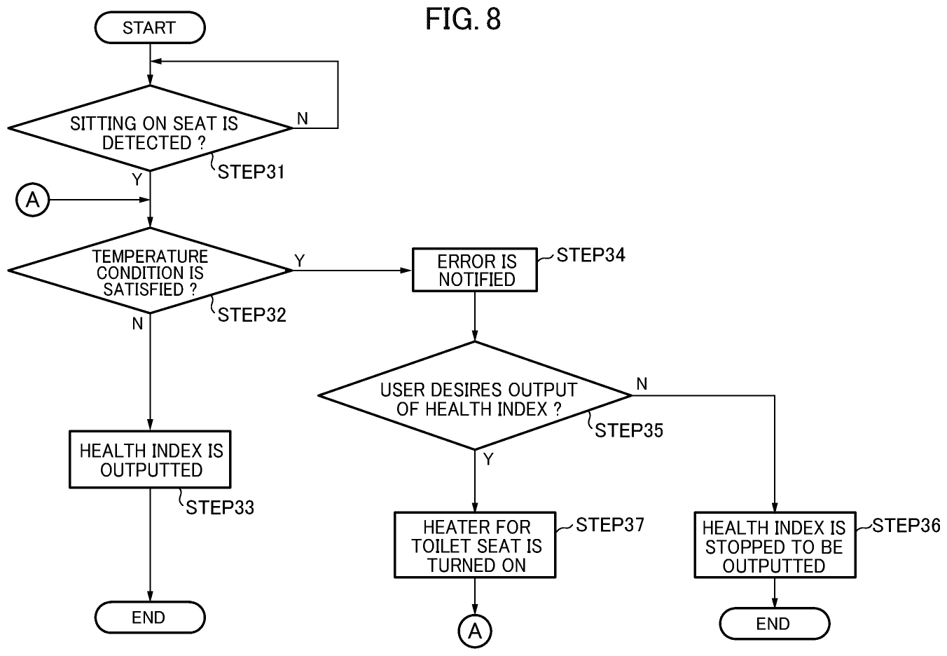

A third operation example of the toilet system 10 as described above is explained with reference to FIG. 8. In the third operation example as well, the controller 70 is configured to determine whether the user sits (continues to sit) on the seat surface 21, based on the measurement results of the capacitance sensor 50 (an example of a seat sensor), as a seat occupancy determining part. In addition, in the third operation example as well, the controller 70 is configured to determine whether the temperature state of the seat surface 21 is not higher than 20° C. (an example of a first threshold value and an example of a second threshold value), based on the measurement results of the temperature thermistor 65 (an example of a temperature sensor), as a temperature determining part. Furthermore, in the third operation example as well, when it is determined that the temperature state of the seat surface 21 is equal to or lower than 20° C. (not higher than 20° C.), the controller 70 is configured to cause the communication part 75 (health index output part) to output error information and/or to stop outputting the health index, as an operation change instruction part.

On the other hand, in the third operation example, when it is determined that the temperature state of the seat surface 21 is equal to or lower than 20° C. (not higher than 20° C.) and a storage part 97 (which may be provided in the main unit 12 (see FIG. 3) or may be a storage unit in the remote controller 80 or the external device (such as the portable terminal of the user) connected via the communication part 75) stores information that the user desires an output of the health index, the controller 70 is configured to forcibly drive the heater wire 30 (an example of a heater) and/or to raise a set temperature thereof (as a heater controller).

### Third Operation Example: Operation

With reference to FIG. 8, the controller 70 determines whether a user sits on the seat surface 21 or not (STEP 31). When it is determined that no user has sat on the seat surface 21 yet (NO at STEP 31), this determining step is repeatedly performed.

When it is determined that a user has sat on the seat surface 21 (YES at STEP 31), the controller 70 determines whether the temperature state of the seat surface 21 is not higher than 20° C. (an example of a second threshold value) (STEP 32). In parallel, a calculation process for a plurality of health indices of the user is started by the health index calculator 60 based on measurement results of the laser sensor 40.

When it is determined that the temperature state of the seat surface 21 is higher than 20° C. (NO at STEP 32), a normal operation is carried out. That is to say, the communication part 75 transmits the health indices of the user calculated by the health index calculator 60 to, for example, the remote controller 80 in the toilet room or the portable terminal 85 of the user (STEP 33).

When it is determined that the temperature state of the seat surface 21 is not higher than 20° C. (YES at STEP 32), the controller 70 causes the communication part 75 (health index output part) to output error information (STEP 34). Subsequently, the controller 70 determines whether the storage part 97 stores the information that the user desires the output of the health index (STEP 35).

When it is not determined (confirmed) that the user desires the output of the health index (NO at STEP 35), the controller 70 causes the communication part 75 (health index output part) to stop outputting the health index (STEP 36).

When it is determined (confirmed) that the user desires the output of the health index (YES at STEP 35), via the toilet seat heating unit 12b, the controller 70 forcibly drives the heater wire 30 (an example of a heater) and/or raises a set temperature thereof (STEP 37). Thereafter, the operation goes back to STEP 32.

When it is determined that the temperature state of the seat surface 21 is higher than 20° C. (NO at STEP 32), it is preferable that the output signal from the temperature thermistor 65 (an example of a temperature sensor) is monitored by the controller 70 as to whether the determined temperature condition continues until completion of the measurement by the laser sensor 40, and it is further preferable that the heater configured to heat the seat surface 21 (specifically, the heater wire 30 in the present embodiment) is controlled (by a suitable combination of “turned on”, “powered up” (a set temperature level thereof is raised), “powered down” (the set temperature level is lowered), “turned off” and the like) by the controller 70 if needed depending on the monitoring results.

### Third Operation Example: Effect

According to the third operation example as described above, for example on a cold winter day, when the temperature of the seat surface 21 is equal to or lower than 20° C. and the user desires the output of the health index, if the heater wire 30 has been off, the heater wire 30 is turned on, or if the heater wire 30 has been on but a set temperature (or a set temperature level) of the heater wire 30 has not been sufficient, the set temperature (or the set temperature level) is raised. Thereby, it is effectively inhibited that any health index that is likely to incorrectly reflect the user's actual health status is outputted as correctly reflecting the user's actual health status.

On the other hand, for example on a cold winter day, when the temperature of the seat surface 21 is equal to or lower than 20° C. and the user does not desire the output of the health index, since the health index is not outputted, it is effectively inhibited that unnecessary confusion and/or upset is given to the user who daily takes care of his or her health with reference to the health index obtained via the toilet system 10.

In addition, according to the third operation example as well, since a temperature of the seat surface 21 is directly measured (obtained), accuracy in a temperature determination is higher.

### Third Operation Example: Variant

In the third operation example as well, information used as the basis of temperature determination is not limited to the measurement results of the temperature thermistor 65. For example, a room temperature sensor configured to measure a temperature of the toilet room (an environment temperature around the seat surface 21) may be provided, and if the heater configured to heat the seat surface 21 is off, the controller may determine (estimate) whether the temperature state of the seat surface 21 satisfies the predetermined condition or not based on measurement results of the room temperature sensor.

Alternatively, information used as the basis of temperature determination may be a combination of an operational state of a heater (specifically, heater wire 30 in the present embodiment) configured to heat the seat surface 21 and weather information (in particular, forecasted temperature) at a region including a house or facility in which the toilet system 10 is installed. The weather information may be received by the toilet system 10 via the internet and may be updated at a suitable timing, for example by means of a suitable IOT technology.

Alternatively, information used as the basis of temperature determination may be an operational state of a heater (specifically, the heater wire 30 in the present embodiment) configured to heat the seat surface 21. Specifically, when the heater is on, a set nominal temperature of the heater (for example, a selected nominal temperature when it is possible to select a plurality of level settings of temperature) may be used as the temperature state of the seat surface 21 in temperature determination. Furthermore, when the heater is off, for example depending on an elapsed time since the heater was turned off, the temperature state of the seat surface 21 may be estimated based on a temperature transition curve forecasted from the set nominal temperature of the heater. In this case, when the elapsed time since the heater was turned off is long enough, the other information as described above (the measurement results of the temperature sensor, the measurement results of the room temperature sensor, and/or the weather information) may be used as the basis of determination.

### Third Operation Example: Further Variant

In the third operation example, in parallel with STEP 32, the calculation process for a plurality of health indices of the user is started by the health index calculator 60 based on the measurement results of the laser sensor 40. In addition, when it is determined that the temperature state of the seat surface 21 is higher than 20° C. (NO at STEP 32), it is preferable that the output signal from the temperature thermistor 65 is monitored by the controller 70 as to whether the determined temperature condition continues until completion of the measurement by the laser sensor 40, and it is further preferable that the heater configured to heat the seat surface 21 is controlled by the controller 70 if needed depending on the monitoring results.

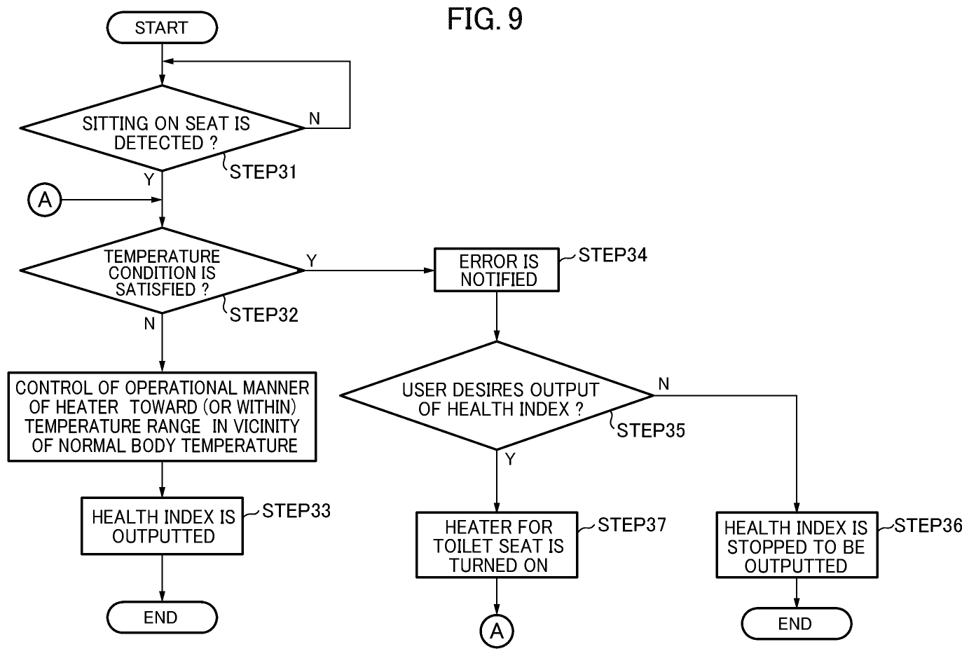

In contrast, in a further variant of the third operation example as shown in FIG. 9, when it is determined that the temperature state of the seat surface 21 is higher than 20° C. (NO at STEP 32), the heater configured to heat the seat surface 21 (specifically, the heater wire 30 in the present embodiment) is controlled (by a suitable combination of “turned on”, “powered up” (a set temperature level thereof is raised), “powered down” (the set temperature level is lowered), “turned off” and the like) by the controller 70, in such a manner that the output signal from the temperature thermistor 65 is brought closer to or maintained within a temperature range in the vicinity of a normal body temperature (skin temperature) (specifically, about 34° C. to 38° C.). Then, while such a control of the operational manner of the heater toward (or within) the temperature range in the vicinity of a normal body temperature is performed, the measurement by the laser sensor 40 is performed, and the calculation process for a plurality of health indices of the user is started by the health index calculator 60 based on the measurement results of the laser sensor 40.

According to the further variant of the third operation example as described above, while the laser sensor 40 measures the blood flow conditions (physical quantity), the controller 70 controls the operational manner of the heater so that the temperature state of the seat surface 21 on which the user sits can be brought close to or maintained within the temperature range in the vicinity of a normal body temperature.

Thereby, it is effectively inhibited that the blood flow conditions of the user who sits on the toilet seat 20 is affected (changed) by the seat surface 21 whose temperature is lower than the user's body temperature, which may happen when the operational manner of the heater is too weak for example in comparison with the room temperature of the toilet room, and that the blood flow conditions of the user who sits on the toilet seat 20 is affected (changed) by the seat surface 21 whose temperature is temporarily higher than the user's body temperature, which may happen when the operational manner of the heater is too strong for example in comparison with the room temperature of the toilet room.

Herein, while the heater's control toward (or within) the temperature range in the vicinity of a normal body temperature is performed as described above, even when a manual function capable of changing the set temperature of the heater is provided on the remote controller 80 in the toilet room or on the portable terminal 85 of the user, it is preferable that such a manual function is interrupted (for example, such a manual input is not accepted).

Supplementary Explanation about Program

The above various functions of the health index calculator 60, the controller 70 and the communication part 75 may be achieved by a microcomputer and the like which executes a corresponding program. Such a program, and a storage medium storing such a program, should be also protected by the present patent application.

For example, a program according to an aspect of the present invention is a program for providing a health index by using the toilet system 10, the toilet system including: the toilet seat 20 having the seat surface 21 on which a user is to sit; and the laser sensor 40 provided in the toilet seat 20, configured to measure a physical quantity which reflects blood flow conditions of the user; the program being capable of perform, when executed by a computer: a health index calculating step of calculating a health index of the user based on measurement results of the laser sensor 40; a health index outputting step of outputting the health index of the user which has been calculated by the health index calculating step; a temperature determining step of determining whether a temperature state of the seat surface 21 satisfies a predetermined condition or not; and an operation change instructing step of changing an operation during the health index calculating step and/or an operation during the health index outputting step when it is determined that the temperature state of the seat surface 21 satisfies the predetermined condition by the temperature determining step.

Alternatively, a program according to another aspect of the present invention is a program for providing a health index by using the toilet system 10, the toilet system including: the toilet seat 20 having the seat surface 21 on which a user is to sit; the laser sensor 40 provided in the toilet seat 20, configured to measure a physical quantity which reflects blood flow conditions of the user; and the heater wire 30 configured to heat the seat surface 21; the program being capable of perform, when executed by a computer: a health index calculating step of calculating a health index of the user based on measurement results of the laser sensor 40; a health index outputting step of outputting the health index of the user which has been calculated by the health index calculating step; a temperature determining step of determining whether a temperature state of the seat surface 21 satisfies a predetermined condition or not; and a heater controlling step of changing an operational manner of the heater wire 30 when it is determined that the temperature state of the seat surface 21 satisfies the predetermined condition by the temperature determining step.

Alternatively, a program according to a further other aspect of the present invention is a program for providing a health index by using the toilet system 10, the toilet system including: the toilet seat 20 having the seat surface 21 on which a user is to sit; the laser sensor 40 provided in the toilet seat 20, configured to measure a physical quantity which reflects blood flow conditions of the user; the storage part 97 configured to store information as to whether the user desires an output of the health index or not; and the heater wire 30 configured to heat the seat surface 21; the program being capable of perform, when executed by a computer: a health index calculating step of calculating a health index of the user based on measurement results of the laser sensor 40; a health index outputting step of outputting the health index of the user which has been calculated by the health index calculating step; a temperature determining step of determining whether a temperature state of the seat surface 21 satisfies a predetermined condition or not; and a heater controlling step of changing an operational manner of the heater wire 30 when it is determined that the temperature state of the seat surface 21 satisfies the predetermined condition by the temperature determining step and the storage part 97 stores the information that the user desires the output of the health index.

What is claimed is:

1. A toilet system comprising:
a toilet seat having a seat surface on which a user is to sit;
a sensor provided in or on the toilet seat, configured to measure a physical quantity which reflects blood flow conditions of the user;
a health index calculator configured to calculate a health index of the user based on measurement results of the sensor;
a health index output part configured to output the health index of the user which has been calculated by the health index calculator;
a temperature determining part configured to determine whether a temperature state of the seat surface satisfies a predetermined condition or not, and
an operation change instruction part configured to change an operation of the health index calculator and/or an operation of the health index output part when the temperature determining part determines that the temperature state of the seat surface satisfies the predetermined condition.

2. The toilet system according to claim 1, wherein
the temperature determining part is configured to determine whether the temperature state of the seat surface is equal to or lower than a predetermined first threshold value or not, and
the operation change instruction part is configured to stop an output operation of the health index output part when the temperature determining part determines that the temperature state of the seat surface is equal to or lower than the first threshold value.

3. The toilet system according to claim 1, wherein
the temperature determining part is configured to determine whether the temperature state of the seat surface is equal to or lower than a predetermined second threshold value or not, and
the operation change instruction part is configured to cause the health index output part to output error information when the temperature determining part determines that the temperature state of the seat surface is equal to or lower than the second threshold value.

4. The toilet system according to claim 1, wherein
the temperature determining part is configured to determine whether the temperature state of the seat surface is within a predetermined third range or not, and
the operation change instruction part is configured to modify a calculation operation of the health index calculator when the temperature determining part determines that the temperature state of the seat surface is within the predetermined third range.

5. The toilet system according to claim 1, further comprising
a temperature sensor configured to measure a temperature of the seat surface,
wherein the temperature determining part is configured to determine whether the temperature state of the seat surface satisfies the predetermined condition or not, based on measurement results of the temperature sensor.

6. The toilet system according to claim 1, further comprising
a room temperature sensor configured to measure an environment temperature around the seat surface,
wherein the temperature determining part is configured to determine whether the temperature state of the seat surface satisfies the predetermined condition or not, based on measurement results of the room temperature sensor.

7. The toilet system according to claim 1, wherein
the temperature determining part is configured to determine whether the temperature state of the seat surface satisfies the predetermined condition or not, based on weather information.

8. The toilet system according to claim 1, further comprising
a heater configured to heat the seat surface,
wherein the temperature determining part is configured to determine whether the temperature state of the seat surface satisfies the predetermined condition or not, based on an operational state of the heater.

9. The toilet system according to claim 1, further comprising
a timer configured to measure a seating time during which the user continues to sit on the seat surface,
wherein the temperature determining part is configured to determine whether the temperature state of the seat surface satisfies the predetermined condition or not, based on measurement results of the timer.

10. A toilet system comprising:
a toilet seat having a seat surface on which a user is to sit;
a sensor provided in or on the toilet seat, configured to measure a physical quantity which reflects blood flow conditions of the user;
a health index calculator configured to calculate a health index of the user based on measurement results of the sensor;
a health index output part configured to output the health index of the user which has been calculated by the health index calculator;
a temperature determining part configured to determine whether a temperature state of the seat surface satisfies a predetermined condition or not,
a heater configured to heat the seat surface,
a storage part configured to store information as to whether the user desires an output of the health index or not, and
a heater controller configured to change an operational manner of the heater when the temperature determining part determines that the temperature state of the seat surface satisfies the predetermined condition, and the storage part stores information that the user desires the output of the health index.

11. A method of providing a health index by using a toilet system comprising:
a toilet seat having a seat surface on which a user is to sit; and
a sensor provided in or on the toilet seat, configured to measure a physical quantity which reflects blood flow conditions of the user;
the method comprising:
a health index calculating step of calculating a health index of the user based on measurement results of the sensor;
a health index outputting step of outputting the health index of the user which has been calculated by the health index calculating step;
a temperature determining step of determining whether a temperature state of the seat surface satisfies a predetermined condition or not, and
an operation change instructing step of changing an operation during the health index calculating step and/or an operation during the health index outputting step when it is determined that the temperature state of the seat surface satisfies the predetermined condition by the temperature determining step.

12. A program for providing a health index by using a toilet system comprising:
a toilet seat having a seat surface on which a user is to sit; and
a sensor provided in or on the toilet seat, configured to measure a physical quantity which reflects blood flow conditions of the user;
the program is capable of perform, when executed by a computer:
a health index calculating step of calculating a health index of the user based on measurement results of the sensor;
a health index outputting step of outputting the health index of the user which has been calculated by the health index calculating step;
a temperature determining step of determining whether a temperature state of the seat surface satisfies a predetermined condition or not; and
an operation change instructing step of changing an operation during the health index calculating step and/or an operation during the health index outputting step when it is determined that the temperature state of the seat surface satisfies the predetermined condition by the temperature determining step.

13. A method of providing a health index by using a toilet system comprising:
a toilet seat having a seat surface on which a user is to sit;
a sensor provided in or on the toilet seat, configured to measure a physical quantity which reflects blood flow conditions of the user;
a heater configured to heat the seat surface,
a storage part configured to store information as to whether the user desires an output of the health index or not,
the method comprising:
a health index calculating step of calculating a health index of the user based on measurement results of the sensor;
a health index outputting step of outputting the health index of the user which has been calculated by the health index calculating step;
a temperature determining step of determining whether a temperature state of the seat surface satisfies a predetermined condition or not; and
a heater controlling step of changing an operational manner of the heater when it is determined that the temperature state of the seat surface satisfies the predetermined condition by the temperature determining step and the storage part stores information that the user desires the output of the health index.

14. A program for providing a health index by using a toilet system comprising:
a toilet seat having a seat surface on which a user is to sit;
a sensor provided in or on the toilet seat, configured to measure a physical quantity which reflects blood flow conditions of the user;
a heater configured to heat the seat surface,
a storage part configured to store information as to whether the user desires an output of the health index or not,
the program is capable of perform, when executed by a computer:
a health index calculating step of calculating a health index of the user based on measurement results of the sensor;
a health index outputting step of outputting the health index of the user which has been calculated by the health index calculating step;
a temperature determining step of determining whether a temperature state of the seat surface satisfies a predetermined condition or not; and
a heater controlling step of changing an operational manner of the heater when it is determined that the temperature state of the seat surface satisfies the predetermined condition by the temperature determining step and the storage part stores information that the user desires the output of the health index.

15. A toilet system comprising:
a toilet seat having a seat surface on which a user is to sit;
a sensor provided in or on the toilet seat, configured to measure a physical quantity which reflects blood flow conditions of the user;
a heater configured to heat the seat surface; and
a heater controller configured to control an operational manner of the heater while the sensor measures the physical quantity.
